(19) United States
(12) Patent Application Publication
(10) Pub. No.: US 2008/0249388 A1
(43) Pub. Date: Oct. 9, 2008

(54) SYSTEMS AND METHODS FOR COOLING OF INTRAVENOUS FLUID AND MONITORING OF IN VIVO CHARACTERISTICS

(75) Inventor: Paul KUMHYR, Wendell, NC (US)

Correspondence Address: LATHROP & GAGE LC, 4845 PEARL EAST CIRCLE, SUITE 300, BOULDER, CO, 80301, US

(73) Assignee: THINKVILLAGE, LLC, Boulder, CO (US)

(21) Appl. No.: 12/098,930

(22) Filed: Apr. 7, 2008

Related U.S. Application Data
(60) Provisional application No. 60/910,500, filed on Apr. 6, 2007.

(51) Int. Cl. A61B 5/00; A61F 7/00

(52) U.S. Cl. 600/368; 604/113

(57) ABSTRACT

Intravenous sensor systems and methods for determining in vivo characteristics are disclosed, as well as systems for cooling bags of intravenous fluid. The intravenous sensor systems include a probe configured for placement in a peripheral vein. The probe includes one or more sensors for obtaining sensed information, such as body temperature, blood oxygen saturation level and blood pressure. An interface unit determines the in vivo characteristics based upon sensed information received from the one or more sensors. The determined in vivo characteristics may be displayed on the interface unit and/or transmitted to a remote computer.

### RELATED APPLICATIONS

This application claims priority to U.S. Provisional Application Ser. No. 60/910,500, filed Apr. 6, 2007, and incorporated herein by reference.

### BACKGROUND

Cardiac arrest disrupts blood supply to a patient's organs and causes widespread oxygen depletion. Such oxygen depletion impacts the brain, which utilizes nearly 20% of circulating blood, more severely than other organs. Even when normal cardiac rhythm resumes, there is competition among the organs for oxygenated blood, and cerebral ischemia and brain damage continue to accrue.

It has recently been discovered that hypothermia therapy may limit damage to the brain during recovery from a cardiac arrest episode. The beneficial effects of hypothermia therapy are believed to result from decreased oxygen demand by the brain, due to slowed metabolism of brain cells, and reduction of swelling.

Some methods for inducing hypothermia, such as immersing a patient in ice, using cooling blankets and perfusing organs with cooled intravenous fluids (e.g., saline, plasma, whole blood, etc.), may be performed by emergency response personnel or paramedics in the field. As induced hypothermia is most beneficial when provided within sixty to ninety minutes of a cardiac arrest episode, these methods are particularly useful. Other methods of inducing hypothermia, such as cooling blood as it flows past a heat exchanging catheter in the inferior vena cava, require surgical procedures that are less practical.

Body temperature must be closely monitored during an induced hypothermia procedure. Typically, monitoring is performed intermittently with a traditional thermometer, e.g., mercury, alcohol or infrared tympanic. However, traditional thermometers do not provide continuous, precise data and rapid changes in body temperature may go undetected.

### SUMMARY

In an embodiment, an intravenous sensor system monitors in vivo characteristics. A probe includes a sensor housing with one or more sensors for sensing in vivo characteristics. An interface unit has a processor and a display. The interface unit determines the in vivo characteristics based upon sensed information received from the probe and displays the in vivo characteristics on the display.

In another embodiment, a method monitors in vivo characteristics. A probe of an intravenous sensor system is inserted into a patient's peripheral vein. An interface unit of the intravenous sensor system is coupled to the probe. The in vivo characteristics are determined from sensed information received from one or more sensors within the probe.

In another embodiment, an intravenous sensor system monitors in vivo characteristics. A probe containing one or more sensors for sensing in vivo characteristics is configured and placed in a peripheral vein of a patent. An interface device is connected to the probe to receive sensed information from the sensors and relay the sensed information to a computer.

In another embodiment, a system cools a bag of intravenous fluid. An insulated receptacle receives the bag of intravenous fluid and a cooling device reduces the temperature of the intravenous fluid within the insulated receptacle.

In another embodiment, a method cools a bag of intravenous fluid. The bag of intravenous fluid is inserted into an insulated receptacle and a cooling device is activated to reduce the temperature of the intravenous fluid within the insulated receptacle.

### BRIEF DESCRIPTION OF THE FIGURES

FIG. 1 shows an exemplary probe of an intravenous sensor system, in an embodiment.

FIG. 2 shows an exemplary interface unit of an intravenous sensor system, in an embodiment.

FIG. 3 shows a schematic of an exemplary intravenous sensor system utilizing the probe of FIG. 1 and the interface unit of FIG. 2.

FIG. 4A shows the sensor housing of the probe of FIG. 1 in further detail.

FIG. 4B shows the probe of FIG. 1 coupled to the interface unit of FIG. 2.

FIG. 5 shows an exemplary probe with a remote interface device, in an embodiment.

FIG. 6 shows an intravenous sensor system inserted into an intravenous catheter, in an embodiment.

FIG. 7 is a flowchart illustrating one exemplary process for monitoring in vivo characteristics, in an embodiment.

FIGS. 8A, 8B and 8C show an exemplary system for cooling intravenous fluid, in an embodiment.

FIG. 9 shows a schematic of one exemplary temperature controlled intravenous fluid cooling system, in an embodiment.

### DETAILED DESCRIPTION OF THE FIGURES

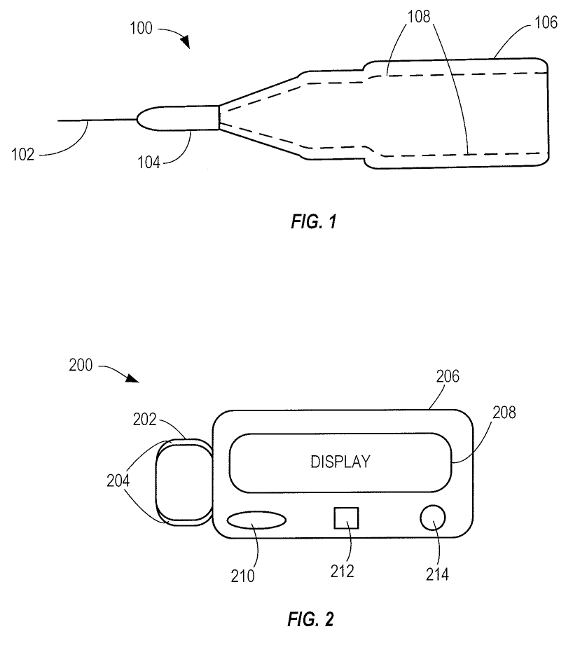

FIG. 1 shows a probe 100 of an intravenous sensor system for in vivo monitoring. Probe 100 includes a needle 102, a sensor housing 104 and a connector 106. Sensor housing 104 may, for example, be a flexible cavity fabricated of biocompatible plastic or an inflexible cavity fabricated of metal, metal alloy or biocompatible plastic. Sensor housing 104 contains one or more sensors for sensing in vivo characteristics, which may for example include body temperature, blood oxygen saturation level (“oxygen level”) and blood pressure. Two or more data paths 108 (e.g., electrodes and/or optical fibers) are disposed along an inside wall of connector 106. Data paths 108 communicate sensed information from the one or more sensors of sensor housing 104 to an interface unit 200, shown in FIG. 2.

FIG. 2 shows an interface unit 200 of an intravenous sensor system for in vivo monitoring. Interface unit 200 includes a connector 202 and a body 206. Connector 202 couples with connector 106 of probe 100 to connect interface unit 200 to probe 100. Connector 202 includes two or more data paths 204 (e.g., electrodes and/or optical fibers) that couple with data paths 108 of probe 100 to receive the sensed information from the one or more sensors of sensor housing 104. Body 206 includes a display 208, a speaker 210, a visual indicator 212 and an input device 214. Display 208 is, for example, an LCD display. Visual indicator 212 may, for example, be a light-emitting diode (LED). Input device 214 may include a keypad or buttons.

It will be appreciated that, in an embodiment, connector 202 may be absent wherein data paths 204 alone may provide sufficient structural integrity to couple with connector 106 of probe 100.

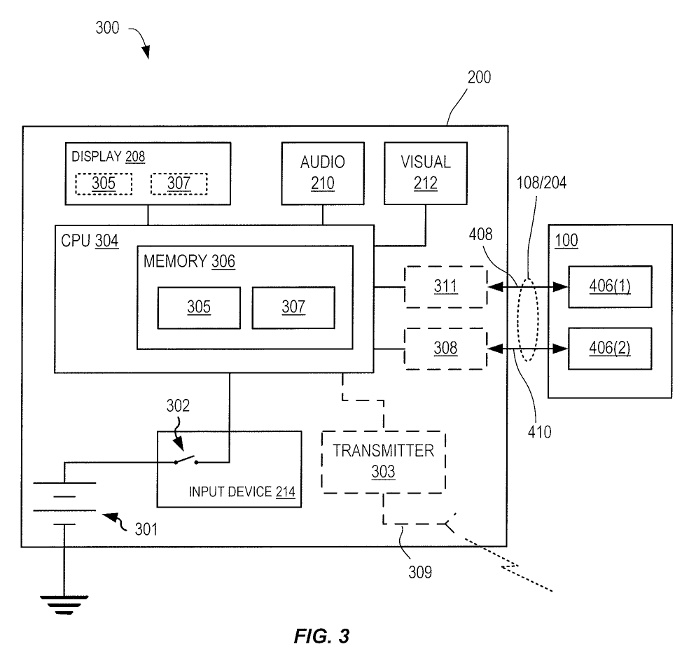

FIG. 3 is a schematic showing one exemplary intravenous sensor system 300 including probe 100 of FIG. 1 and interface unit 200 of FIG. 2. In an embodiment, intravenous sensor system 300 is powered by a battery 301 when switch 302 is closed, e.g., by pressing a button of input device 214. Power may alternatively be provided to system 300 from other suitable power sources, such as 50/60 Hz power, solar cells and the like, without departing from the scope hereof.

A central processing unit (CPU) 304 receives sensed information, via data paths 108 and 204, from one or more sensors of sensor housing 104. CPU 304 stores this sensed information within a memory 306 of CPU 304. CPU 304 may represent one or more of a microprocessor, a microcontroller or an application specific integrated circuit (“ASIC”). CPU 304 controls operation of interface unit 200 and processes the sensed information received from the one or more sensors of sensor housing 104 to determine in vivo characteristics 305, such as one or more of body temperature, oxygen level, and blood pressure. These in vivo characteristics 305 may be displayed on display 208. Memory 306 may also store one or more threshold ranges 307 for each in vivo characteristic. Threshold ranges 307 may also represent a single value threshold without departing from the scope hereof. Threshold ranges 307 are set by the user operating input device 214 while viewing threshold ranges 307 on display 208, for example. If a determined in vivo characteristic 305 falls outside of the set threshold range 307, and an alarm feature has been enabled by the user, CPU 304 may control audio indicator 210 to generate a sound and/or control visual indicator 212 to generate a visible signal. Once activated, indicators 210 and 212 are deactivated by the user pressing a button on input device 214, for example. In an embodiment, audio and/or visual indicators 210 and 212 are operated when power of battery 301 is low to indicate that battery 301 should be changed. Display 208 may also indicate battery status without departing from the scope hereof.

In an embodiment, interface unit 200 includes a transmitter 303 and an antenna 309 that transmits sensed in vivo characteristics 305 to a remote receiver (not shown), such as within a remote work station.

In an embodiment, an optical module 308, shown within interface unit 200, includes one or more wavelength-specific LEDs and one or more photodiodes for sensing oxygen level and/or blood pressure, as described below. In particular, optical module 308 provides an optical/electrical interface for providing an electrical signal, indicative of one or more of blood pressure and/or blood oxygen level, for input to CPU 304. CPU 304 may include one or more analog to digital (A/D) converters for digitizing these received signals.

In an embodiment, an electronic conditioning module 311, shown within interface unit 200, includes one ore more electronic components for providing power to sensors 406 and for conditioning sensed information received from sensors 406. For example, electronic signals from sensor 406 are conditioned to make them suitable for input to CPU 304.

Body temperature, oxygen level and blood pressure threshold ranges 307 may be specified with upper and/or lower limits such that an audio and/or visual indicator operates when any one or more of sensed body temperature, oxygen level and blood pressure falls outside these specified ranges. For example, the user may specify upper and lower temperature limits such that an alarm occurs when the sensed temperature falls below the lower limit or rises above the upper limit. On one example, these limits may be between 33 and 35 degrees Celsius (mild hypothermia), or between 28 and 33 degrees Celsius (moderate hypothermia), or between 24 and 28 degrees Celsius (severe hypothermia).

When hypothermia is induced via perfusion, it is preferred that the patient's body temperature stays within the range of 32 to 34 degrees Celsius. In one example of operation, probe 100 is inserted into one of the patient's veins such that system 300 operates to measure the patient's in vivo characteristics (e.g., sensed in vivo characteristics 305). If the sensed temperature rises above 34 degrees Celsius, audio indicator 210 and/or visual indicator 212 may be activated to alert a medical provider that additional cold fluids are needed. If the measured body temperature falls below 32 degrees Celsius, an indicator 210, 212 may alert a medical provider that administration of cold intravenous fluids should be reduced (e.g., to a minimum flow rate of 30 mL/hr). In an embodiment, system 300 includes an automated intravenous fluid delivery system (not shown) such that CPU 304 controls delivery of cooled intravenous fluid based upon measured temperature. For example, CPU 304 may communicate wirelessly with the automated intravenous fluid delivery system using transmitter 303 and antenna 309. Alternatively, a wired connection may be used to control the automated intravenous fluid delivery system.

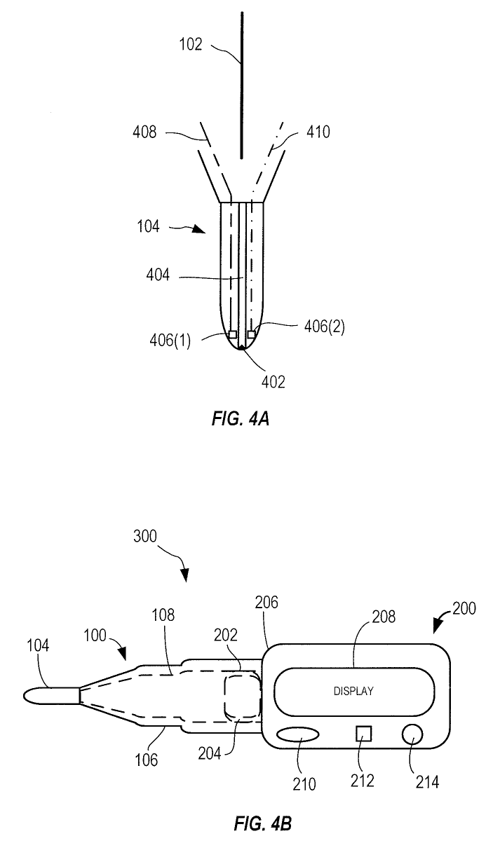

FIG. 4A shows needle 102 and sensor housing 104 of system 300 in further detail. In operation, needle 102 guides sensor housing 104 of probe 100 through a patient's skin and into a peripheral vein. A peripheral vein is for example any vein not in the chest or the abdomen. Ann and hand veins are typically used for monitoring, although leg and foot veins may also be used. Typically, the basilic vein or cubital vein of the arm, or the external jugular vein of the neck is used. Once probe 100 is inserted into the selected vein, needle 102 is removed or retracted through a channel 404 that has a one-way valve 402 at a distal end of sensor housing 104 to prevent blood from entering sensor housing 104. Sensor housing 104 remains in the vein after removal or retraction of needle 102, and connector 106 is coupled with connector 202 of interface unit 200. Upon connection of interface unit 200 and probe 100, visual indicator 212 and/or display 208 may illuminate indicating that system 300 is operational. A completed assembly of system 300 is shown in FIG. 4B. Portions of system 300 located outside of the patient's body may be taped in place or secured with a self-adhesive dressing.

It will be appreciated that interface unit 200 does not have direct contact with body fluids, such as blood. Interface unit 200 may therefore be reused with other probes 100 without risk of cross-contamination.

Since the intravenous sensor system remains in the patient's vein, in vivo characteristics 305 may be continuously monitored or monitored periodically (e.g., once a minute, once a number of minutes, once an hour, once a number of hours, twice a day, once a day and once a number of days). Through use of system 300, in vivo characteristics 305 may be obtained, without disturbing the patient, by viewing display 208 of interface unit 200 or by receiving periodically transmitted, via transmitter 303 and antenna 309, sensed in vivo characteristics 305 at a remote location, such as a nurses' station.

Sensor housing 104 is shown with two sensors 406(1) and 406(2) for clarity of illustration. Sensor housing 104 may include more or fewer sensors 406 without departing from the scope hereof. Sensor 406 operates to monitor one or more of body temperature, oxygen level and blood pressure. Sensor 406(1) may be a thermocouple or thermistor for measuring the temperature of blood passing outside sensor housing 104 (i.e., by sensing heat transfer through the wall of sensor housing 104). Sensor 406(1) may form at least a portion of sensor housing 104 wall thereby having direct contact with the patient's blood. System 300 may include conditioning electronics 311 that electrically connect, via electrodes 408, with sensor 406(1) to measure temperature.

Where sensor 406(2) is an optical sensor, blood oxygen level may be sensed. In one example, an optical fiber 410 sequentially delivers two wavelengths of light, 660 nm from a red LED and one of 905 nm, 910 nm and 940 nm from an infrared LED, from within optical module 308. Absorption at these wavelengths differs significantly between oxyhemoglobin and its deoxygenated form. Thus, a ratio of oxygenated to deoxygenated hemoglobin may be determined within CPU 304. In an embodiment, a reflectance configuration of LEDs and a photodiode detector may be utilized. Optical fiber 410 delivers light generated by one or more LEDs within optical module 308 to sensor 406(2) where the light is emitted from sensor housing 104 to the patient's blood. Optical fiber 410 also functions to return light reflected by the patient's blood to a photodiode within optical module 308, where the reflected light is detected and transduced into an electrical signal that is received and processed by CPU 304. Sensor housing 104 may be fabricated of a material that is transparent in the visible and near infrared wavelength range generated by optical module 308. Suitable fabrication materials include, for example, quartz, polystyrene, polycarbonate and polypropylene.

In an embodiment, sensor 406(1) represents a blood pressure sensor formed from a piezoelectric material that is either directly in contact with blood, or in contact with a flexible membrane that translates blood pressure changes to the piezoelectric material. In another embodiment, sensor 406(2) represents a blood pressure sensor that is an optical sensor acting as an interferometer to detect changes in a silicon or flexible polymer (e.g., polyurethane, polystyrene) diaphragm. The diaphragm is in contact with the blood (i.e., the diaphragm forms part of the sensor housing wall) and variations in pressure cause the diaphragm to flex, thereby altering the cavity length of the interferometer. Thus, the unit may provide combined blood-pressure sensing and oxygen level measurements by using the same LEDs and photodiode detector within optical module 308.

It is appreciated that sensors 406 are small enough to fit within sensor housing 104 which is to be inserted within a patient's vein while permitting continued blood flow within that vein. The size of sensor housing 104 is preferably between 14 and 22 gauge (i.e., the diameter of sensor housing 104 is between 25-65 thousandths of an inch).

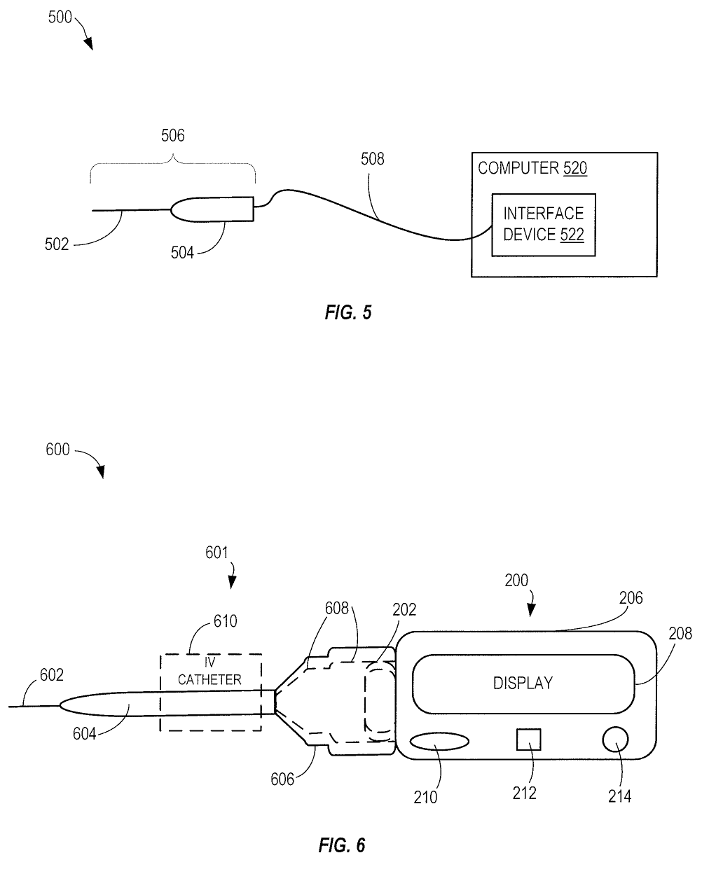

FIG. 5 shows one exemplary intravenous sensor system 500 for in vivo monitoring. System 500 includes a probe 506 that connects, via a data path 508 (e.g., electrodes and/or optical fibers), to an interface device 522 within a computer 520. Computer 520 is for example a nursing station and includes software appropriate for control and monitoring of probe 506 via interface device 522. Interface device 522 includes electronic and optical components for controlling and utilizing one or more sensors within probe 506. Interface device 522 may be external to computer 520 without departing from the scope hereof. For example, interface device 522 may connect to computer 520 via a standard computer communication port (e.g., USB, serial, parallel, firewire, etc.). Probe 506 is similar to probe 100, FIG. 1, and includes a needle 502, a sensor housing 504 and one or more sensors (not shown). Data path 508 represents one or more electrodes and/or optical fibers that may be grouped into a single cable. Probe 506 senses in vivo characteristics such as one or more of body temperature, oxygen level and blood pressure. Data path 508 conveys sensed information from sensors within sensor housing 504 to computer 520 via interface device 522. Computer 520 may include a display, a keyboard and a mouse for control of probe 506 and display of sensed in vivo characteristics. Software of computer 520 may provide alarm functionality based upon one or more user defined ranges for sensed in vivo characteristics. In an embodiment, computer 520 may be interface unit 200.

FIG. 6 stows one exemplary intravenous sensor system 600 for use in conjunction with an intravenous catheter 61 0. For example, where intravenous catheter 610 is used to deliver intravenous fluids and medications to a peripheral vein of a patient, intravenous catheter 610 is inserted into a patient s peripheral vein using a needle which is subsequently removed from the catheter. Sensor housing 604 of probe 601 is then inserted through catheter 610 and into the patient's vein to gain access to blood flowing through the vein. A needle 602 of probe 601 may be used to guide sensor housing 604 through intravenous catheter 610 and then subsequently removed or withdrawn, as previously described. Sensor housing 604 remains inside the vein, and connector 606 connects to interface unit 200 via connector 202. Once connector 606 and connector 202 are connected, visual indicator 212 may illuminate to indicate that system 600 is operational.

Probes 100, 506 and 601 may be utilized without a need for special surgical procedures and advanced imaging to guide the probes into place. The systems and methods described herein may thus be used in the field to provide continuous, precise monitoring of in vivo characteristics.

It will be appreciated that system 500 of FIG. 5 may be inserted into an intravenous catheter in a manner similar to that described for system 600 of FIG. 6.

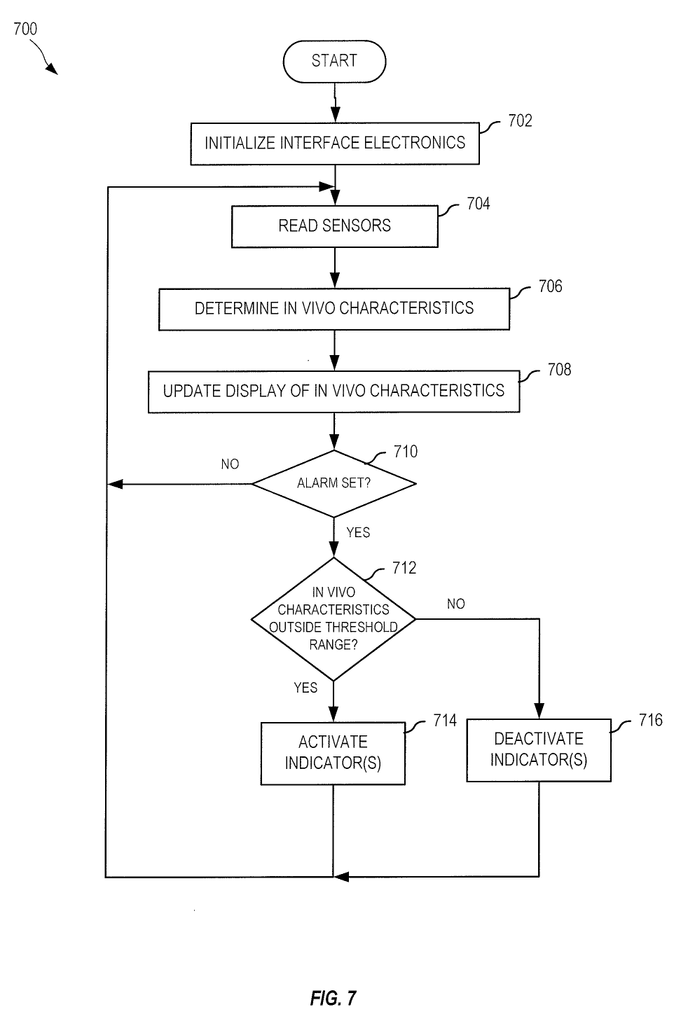

FIG. 7 is a flowchart illustrating one exemplary process 700 for monitoring in vivo characteristics. Process 700 is, for example, implemented within CPU 304 of interface unit 200. In step 702, process 700 initializes interface electronics. In one example of step 702, CPU 304 clears memory 306 and initializes optical module 308 and electronic conditioning module 311 to activate sensors 406. In step 704, process 700 reads sensor values. In one example of step 704, CPU 304 samples signal from optical module 308 and electronic conditioning module 311. In step 706, method 700 determines in vivo characteristics. In one example of step 706, CPU 304 utilizes one or more algorithms to convert the sampled values of step 704 into in vivo characteristics 305. In step 708, process 700 updates the display of in vivo characteristics. In one example of step 708, CPU 304 sends in vivo characteristics 305 to display 208. Step 710 is a decision. If, in step 704, process 700 determines that the alarm function is set, process 700 continues with step 712; otherwise process 700 continues with step 704.

Step 712 is a decision. If, in step 712, process 700 determines that the in vivo characteristics determined in step 706 are outside of defined threshold ranges, process 700 continues with step 714; otherwise process 700 continues with step 716. In step 714, process 700 activates indicators. In one example of step 714, CPU 304 activates audio indicator 210 and activates visual indicator 212, thereby providing an indication to the user that sensed characteristics are not within the user specified ranges. Process 700 continues with step 704.

In step 716, process 700 deactivates indicators. In one example of step 716, CPU 304 deactivates audio indicator 210 and deactivates visual indicator 212. Process 700 continues with step 704.

Steps 704 through 716 repeat to maintain operation of interface unit 200. As appreciated, the order of steps 704 through 714 may vary without departing from the scope hereof.

Since induced hypothermia is most beneficial when provided within sixty to ninety minutes of a cardiac arrest episode, and there is limited space in an emergency vehicle to immerse a patient in ice or use a cooling blanket, perfusion with cold fluids is especially useful. However, bags of intravenous fluid are usually stored at room temperature, and use of a separate facility for cooling and storage of additional bags is not space or cost effective.

The present disclosure provides systems and methods for cooling individual bags of intravenous fluid so that cooling may be initiated during transit to or upon arrival at a cardiac arrest scene. Such systems eliminate the space and cost issues related to maintaining cold intravenous fluids.

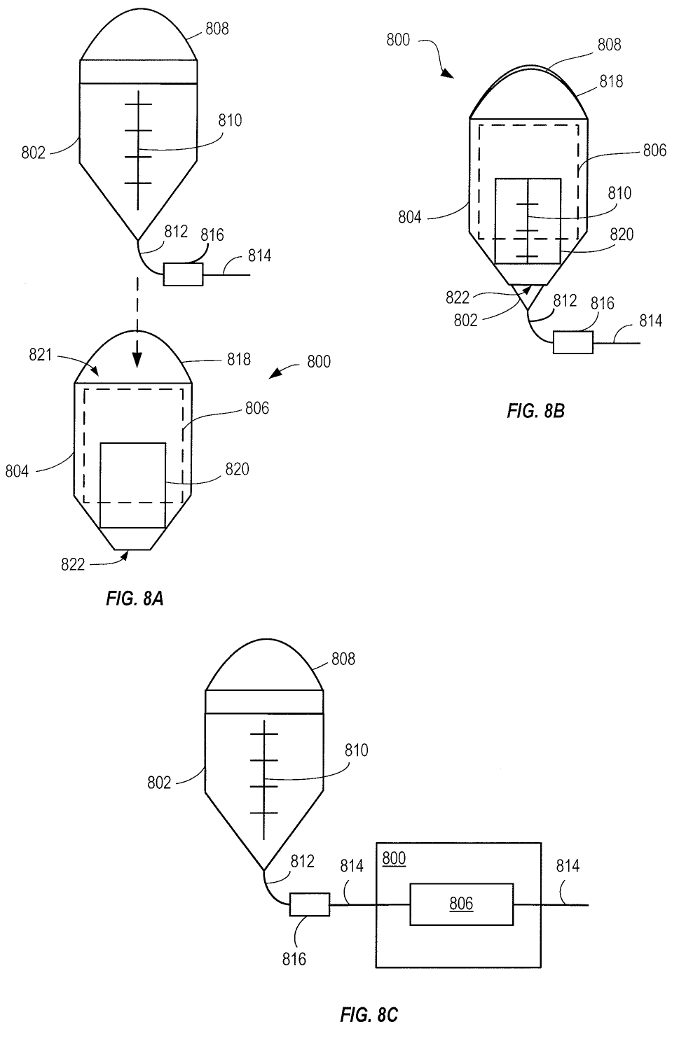

FIGS. 8A and 8B show one exemplary intravenous fluid cooling system 800 for cooling an individual bag 802 of intravenous fluid. Bag 802 may for example contain one liter of saline, plasma, whole blood or the like. Bag 802 is shown with a handle 808, a fluid level indicator 810, medication lines 812, 814, and a medication port 816 (e.g., an intravenous drip). System 800 includes an insulated receptacle 804 for receiving bag 802 (e.g., inserted through a top opening 821 of insulated receptacle 804 as shown by an arrow in FIG. 8A) and a cooling device 806. Cooling device 806 may be a chemical coolant, a compressor based cooling device or a Peltier based cooling device. Insulated receptacle 804 includes a handle 818 and a transparent window 820 configured to allow monitoring of fluid level indicator 810, as shown in FIG. 8B. Insulated receptacle 804 may, for example, be fabricated from neoprene, polyurethane or nitrile rubber foam. An opening 822 at the bottom of insulated receptacle 804 allows medication lines 812, 814 and medication port 816 to pass therethrough.

Chemical coolants, as may be used with cooling device 806 in an embodiment, are commercially available in the form of instant cold packs, which may include calcium chloride, ammonium nitrate and/or urea based products. A pouch containing the chemical coolant may be placed directly into insulated receptacle 804 in direct contact with bag 802. Alternatively, insulated receptacle 804 may contain a pocket for receiving the chemical coolant pouch such that the pouch and the intravenous fluid bag are physically separated from, but in thermal contact with, one another. One disadvantage of chemical coolants is that their temperature is not easily regulated, e.g., using thermostatic control.

In another embodiment, cooling device 806 is Peltier based and is in thermal contact with bag 802 of intravenous fluid. Cooling device 806 may be a thermoelectric plate constructed within insulated receptical 804 such that heat is transferred from within insulated receptacle 804 to air external to insulated receptical 804. Alternatively, a Peltier device in the form of a probe may be inserted directly into the flow of intravenous fluid at medication port 816 to provide cooling of the intravenous fluid during administration thereof.

In another embodiment, cooling device 806 is compressor based and is external to insulated receptacle 804 such that intravenous fluid passes through cooling device 806 during administration of the fluid. For example, intravenous fluid may be pumped through cooling device 806 or may flow as a result of gravity. In one example, one or more of medication lines 812 and 814 pass through or connect to cooling device 806 as shown in FIG. 8C.

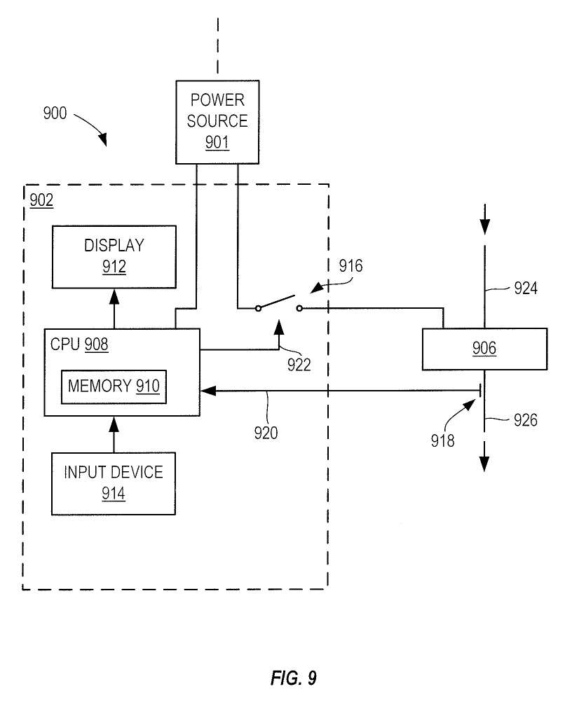

FIG. 9 shows a schematic of one exemplary temperature controlled intravenous fluid cooling system 900. System 900 includes a thermostatic control device 902 and a cooling device 906. Thermostatic control device 902 includes a display 912, a CPU 908, a switch 916 and an input device 914. CPU 908 is, for example, a microprocessor, a microcontroller or an application specific integrated circuit (“ASIC ”) suitable for controlling operation of cooling device 906. CPU 908 may include a memory 910. Thermostatic control device 902 and cooling device 906 are shown powered from a power source 901 external to device 902. Power source 901 may be internal to device 902 without departing from the scope hereof. In one example, power source 901 is a battery. In another example, power source is a power converter (e.g., a transformer) connected to a suitable external power supply, such as external 50/60 Hz AC grid power, a vehicle battery, solar cells, or the like.

Cooling device 906 may represent cooling device 806 of FIGS. 8A, 8B and 8C, such that cooling device 806 has temperature control through device 902. Intravenous fluid flows into cooling device 906 through an inlet tube 924 and out of cooling device 906 through an outlet tube 926. Tubes 924 and 926 may represent one or both of medication lines 812, 814. A temperature sensor 918 senses temperature of cooled intravenous fluid flowing out of cooling device 906. Sensor 918 may be inserted into the flow of intravenous fluid or may attach to tube 926. Where sensor 906 is enclosed within an insulated receptical together with a bag of intravenous fluid to be cooled, such as device 806 within insulated receptical 804, sensor 918 may also be located within the insulated receptical.

CPU 908 operates to determine the temperature of the intravenous fluid based upon a sensed temperature signal received from sensor 918 via a connection 920. CPU 908 may display the determined temperature upon display 912. Memory 910 is used to store at least one temperature threshold value that defines the desired temperature of intravenous fluid output through tube 926. This threshold value may be set by operation of input device 914 by a user, wherein the threshold value is displayed upon display 912 while being selected by the user.

In one example of operation, CPU 908 receives sensed temperature from sensor 918 and determines the temperature of intravenous fluid output from tube 926. If the determined temperature is above the threshold value, CPU 908 controls switch 916 to close, thereby providing power to cooling device 906; with power applied, cooling device 906 cools the intravenous fluid. If the determined temperature is lower than the threshold value, CPU 908 controls switch 916 to open, thereby removing power from cooling device 906; without power cooling device 906 stops cooling the intravenous fluid.

It will be appreciated that changes to system 900 may be made without departing from the scope hereof. For example, thermostatic control device 902 may include a visual indicator (such as on display 912) and an audio indicator to provide the user with additional information regarding the measured temperature of the intravenous fluid and operation of thermostatic control device 902. In another embodiment, thermostatic control device 902 may be disposed with, or integrated within, insulated receptacle 804.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present methods and systems, which, as a matter of language, might be said to fall there between.

What is claimed is:

1. An intravenous sensor system for monitoring in vivo characteristics, comprising:
a probe comprising a sensor housing with one or more sensors for sensing in vivo characteristics; and
an interface unit comprising a CPU and a display, the interface unit determining the in vivo characteristics based upon sensed information received from the probe and displaying the in vivo characteristics on the display.

2. The system of claim 1, the probe further comprising a needle for guiding at least a portion of the probe into a patient's peripheral vein.

3. The system of claim 2, the needle being retractable once the at least a portion of the probe is in the vein, the probe comprising a one-way valve at a distal end to prevent blood from entering the probe.

4. The system of claim 2, the needle being removable once the at least a portion of the probe is in the vein, the probe comprising a one-way valve at a distal end to prevent blood from entering the probe.

5. The system of claim 2, the probe being configured to penetrate an intravenous catheter inserted into the vein, the distal tip of the probe extending beyond the intravenous catheter and into the vein.

6. The system of claim 1, the one or more sensors being selected from the group including a temperature sensor, an oxygen sensor, and a blood pressure sensor.

7. The system of claim 7, wherein the in vivo characteristic comprises one or more of body temperature, oxygen level, and blood pressure.

8. The system of claim 1, the interface unit comprising one or both of an audio indicator and a visual indicator, the CPU activating the one or both indicators if any one of the determined in vivo characteristics exceeds a set threshold.

9. The system of claim 8, the interface unit comprising an input device for receiving input from a user to set the threshold.

10. The system of claim 1, wherein the interface unit comprises a transmitter and an antenna for transmitting the in vivo characteristics to a remote receiver.

11. The system of claim 1, wherein the interface unit comprises a memory for storing the in vivo characteristics.

12. A method for intravenous monitoring of in vivo characteristics, comprising:
inserting a probe of an intravenous sensor system into a patient's peripheral vein;
coupling an interface unit of the intravenous sensor system to the probe; and
determining the in vivo characteristic based upon sensed information received from one or more sensors within the probe.

13. The method of claim 12, further comprising displaying the in vivo characteristic on a display of the interface unit.

14. The method of claim 12, further comprising activating an indicator to alert a user when one or more of the in vivo characteristics fall outside of a threshold range.

15. The method of claim 14, further comprising setting the threshold range via an input device.

16. The method of claim 12, wherein the in vivo characteristic comprises one or more of body temperature, oxygen level, and blood pressure.

17. An intravenous sensor system for in vivo monitoring, comprising:
a probe configured for placement in a peripheral vein of a patent, the probe containing one or more sensors for sensing in vivo characteristics; and
an interface device connected to the probe to receive sensed information from the probe and for relaying the sensed information to a computer.

18. The system of claim 17, the probe comprising a needle for guiding the probe into the peripheral vein.

19. The system of claim 18, wherein the probe comprises a one-way valve that allows the needle to be retracted.

20. The system of claim 17, wherein a portion of the probe is configured to penetrate an intravenous catheter.

21. The system of claim 17, wherein the one or more sensors are selected from the group including a temperature sensor, an oxygen sensor, and a blood pressure sensor.

22. A system for cooling a bag of intravenous fluid, comprising:
an insulated receptacle configured to receive the bag of intravenous fluid; and
a cooling device for reducing the temperature of the intravenous fluid within the insulated receptacle.

23. The system of claim 22, the insulated receptacle having a transparent window for monitoring a level of the intravenous fluid in the bag.

24. The system of claim 22, wherein the cooling device is selected from the group including a chemical coolant, a compressor and a Peltier device.

25. The system of claim 22, further comprising a thermostatic controller for controlling operation of the cooling device based upon sensed temperature of the intravenous fluid.

26. The system of claim 22, further comprising a display for indicating a temperature of the intravenous fluid.

27. A method for cooling a bag of intravenous fluid, comprising:
inserting the bag of intravenous fluid into an insulated receptacle; and
activating a cooling device to reduce the temperature of the intravenous fluid within the insulated receptacle.

28. The method of claim 27, further comprising setting a temperature threshold of a thermostatic controller to control operation of the cooling device to maintain the temperature of the intravenous fluid based upon the set temperature.
